(19) United States
(12) Patent Application Publication
(10) Pub. No.: US 2022/0080099 A1
(43) Pub. Date: Mar. 17, 2022

(54) OPHTHALMIC CURETTE

(71) Applicant: JOHNSON & JOHNSON SURGICAL VISION, INC., Santa Ana, CA (US)

(72) Inventors: Assaf Govari, Haifa (IL); Eran Aharon, Haifa (IL); Vadim Gliner, Haifa (IL); Ilya Sitnitsky, Nahariya (IL); Yehuda Algawi, Binyamina (IL)

(21) Appl. No.: 17/536,174

(22) Filed: Nov. 29, 2021

Related U.S. Application Data
(63) Continuation-in-part of application No. 16/922,200, filed on Jul. 7, 2020.

(51) Int. Cl. A61M 1/00; A61F 9/007

(52) U.S. Cl. CPC A61M 1/774; A61F 9/00736; A61M 2207/00; A61M 2210/0612; A61M 2205/3331

(57) ABSTRACT

In one embodiment, a phacoemulsification system includes a phacoemulsification probe configured to be inserted into an eye, an ophthalmic curette, including a handle, a tube having a proximal end connected to a distal end of the handle, and having a distal tip configured to be inserted into the eye, an irrigation channel extending from the proximal end to the distal tip of the tube, and a pressure sensor disposed at the distal tip of the tube, and configured to be inserted into the eye and provide a signal responsively to intraocular pressure inside the eye, and comprising a sensing surface, and an irrigation-aspiration sub-system coupled with the irrigation channel and configured to convey irrigation fluid along the irrigation channel, and wherein the irrigation channel is shaped, and positioned with respect to the pressure sensor, to direct a flow of the irrigation fluid over the sensing surface of the pressure sensor.

### RELATED APPLICATION INFORMATION

The present application is a Continuation-In-Part of U.S. patent application Ser. No. 16/922,200 of Algawi, et al., filed Jul. 7, 2020, the disclosure of which is hereby incorporated herein by reference.

### FIELD OF THE INVENTION

The present invention relates to medical devices, and in particular to, ophthalmic curettes.

### BACKGROUND

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this a physician may recommend phacoemulsification cataract surgery. Before the procedure the surgeon numbs the area with anesthesia. Then a small incision is made in the cornea of the eye. Fluids are injected into this incision to support the surrounding structures. The anterior surface of the lens capsule is then removed to gain access to the cataract. The surgeon then uses a phacoemulsification probe, which has an ultrasonic handpiece with a titanium or steel needle. The tip of the needle vibrates at ultrasonic frequency to sculpt and emulsify the cataract, while a pump device aspirates particles from the cataract through the tip. The pump is typically controlled with a microprocessor. The pump may be a peristaltic or a venturi type of pump. Aspirated fluids are replaced with irrigation of a balanced salt solution to maintain the anterior chamber of the eye. After removing the cataract with phacoemulsification, the softer outer lens cortex is removed with suction. An intraocular lens (IOL) is introduced into the empty lens capsule. Small struts called haptics help hold the IOL in place. Once correctly implanted the IOL restores the patient's vision.

### SUMMARY

There is provided in accordance with an embodiment of the present disclosure, a phacoemulsification system, including a phacoemulsification probe configured to be inserted into an eye, an ophthalmic curette, including a handle having a distal end, a tube having a proximal end connected with the distal end of the handle, and having a distal tip configured to be inserted into the eye, an irrigation channel extending from the proximal end to the distal tip of the tube, and a pressure sensor including a sensing surface and disposed at the distal tip of the tube, wherein the pressure sensor is configured to be inserted into the eye and provide a signal responsively to intraocular pressure inside the eye, and an irrigation-aspiration sub-system coupled with the irrigation channel and configured to convey irrigation fluid along the irrigation channel, and wherein the irrigation channel is shaped, and positioned with respect to the pressure sensor, to direct a flow of the irrigation fluid over the sensing surface of the pressure sensor.

Further in accordance with an embodiment of the present disclosure the tube has a lumen, the irrigation channel having walls defined by the lumen.

Still further in accordance with an embodiment of the present disclosure the tube has a lumen, the irrigation channel having walls disposed in the lumen.

Additionally in accordance with an embodiment of the present disclosure the pressure sensor is at least partially disposed in the irrigation channel.

Moreover, in accordance with an embodiment of the present disclosure the irrigation channel is disposed in the tube and extends from the proximal end to the distal tip, and wherein the pressure sensor is disposed at least partially in the irrigation channel at the distal tip.

Further in accordance with an embodiment of the present disclosure the tube includes a beveled opening, which extends longitudinally along all of the distal tip of the tube.

Still further in accordance with an embodiment of the present disclosure the beveled opening defines a plane which has an angle in the range of 30 to 70 degrees with a plane perpendicular to a direction of elongation of the tube.

Additionally in accordance with an embodiment of the present disclosure the tube has a minimum length of 2 cm.

Moreover, in accordance with an embodiment of the present disclosure the tube has an outer diameter between 0.1 mm and 0.8 mm.

Further in accordance with an embodiment of the present disclosure the tube has a wall thickness between 0.03 mm and 0.2 mm.

Still further in accordance with an embodiment of the present disclosure the pressure sensor is coated with a waterproof coating.

Additionally in accordance with an embodiment of the present disclosure the waterproof coating is selected from a group consisting of Parylene, silicon, and polyurethane.

Moreover, in accordance with an embodiment of the present disclosure the irrigation-aspiration sub-system is configured to convey irrigation fluid along the irrigation channel at a rate of between 1 and 5 milliliters per minute.

Further in accordance with an embodiment of the present disclosure the irrigation-aspiration sub-system is configured to convey irrigation fluid along the irrigation channel intermittently providing periods of irrigation activity and intervening periods of irrigation inactivity, the system further including a processor configured to sample respective values from the signal provided by the pressure sensor corresponding to time values during the periods of irrigation inactivity, and compute pressure values responsively to the respective sampled values.

Still further in accordance with an embodiment of the present disclosure the irrigation-aspiration sub-system is configured to convey irrigation fluid along the irrigation channel responsively to a modulated irrigation rate providing periods of irrigation activity above a given irrigation rate and intervening periods of irrigation activity below the given irrigation rate, the system further including a processor configured to sample respective values from the signal provided by the pressure sensor corresponding to time values during the periods of irrigation activity below the given irrigation rate, and compute pressure values responsively to the respective sampled values.

Additionally in accordance with an embodiment of the present disclosure, the system includes a processor configured to compute a pressure value responsively to the signal provided by the pressure sensor, and wherein the phacoemulsification probe includes an irrigation line and an aspiration line, the irrigation-aspiration sub-system is coupled with the irrigation line and the aspiration line, and the processor is configured to control the irrigation-aspiration sub-system to adjust at least one of the following responsively to the computed pressure value an aspiration rate of eye fluid from the eye via the aspiration line of the phacoemulsification probe, an irrigation rate of the irrigation fluid to the eye via the irrigation line of the phacoemulsification probe, and an irrigation rate of the irrigation fluid to the eye via the irrigation channel of the ophthalmic curette.

There is also provided in accordance with another embodiment of the present disclosure, an ophthalmic curette apparatus, including a handle having a distal end, a tube having a proximal end connected with the distal end of the handle, and having a distal tip configured to be inserted into an eye, an irrigation channel extending from the proximal end to the distal tip of the tube, and a pressure sensor including a sensing surface and disposed at the distal tip of the tube, wherein the pressure sensor is configured to be inserted into the eye and provide a signal responsively to intraocular pressure inside the eye, wherein the irrigation channel is configured to be coupled with an irrigation-aspiration sub-system to convey irrigation fluid along the irrigation channel, and wherein the irrigation channel is shaped, and positioned with respect to the pressure sensor, to direct a flow of the irrigation fluid over the sensing surface of the pressure sensor.

Moreover, in accordance with an embodiment of the present disclosure the tube has a lumen, the irrigation channel having walls defined by the lumen.

Further in accordance with an embodiment of the present disclosure the tube has a lumen, the irrigation channel having walls disposed in the lumen.

Still further in accordance with an embodiment of the present disclosure the pressure sensor is at least partially disposed in the irrigation channel.

Additionally in accordance with an embodiment of the present disclosure the irrigation channel is disposed in the tube and extends from the proximal end to the distal tip, and wherein the pressure sensor is disposed at least partially in the irrigation channel at the distal tip.

Moreover, in accordance with an embodiment of the present disclosure the tube includes a beveled opening, which extends longitudinally along all of the distal tip of the tube.

Further in accordance with an embodiment of the present disclosure the beveled opening defines a plane which has an angle in the range of 30 to 70 degrees with a plane perpendicular to a direction of elongation of the tube.

Still further in accordance with an embodiment of the present disclosure the tube has a minimum length of 2 cm.

Additionally in accordance with an embodiment of the present disclosure the tube has an outer diameter between 0.1 mm and 0.8 mm.

Moreover, in accordance with an embodiment of the present disclosure the tube has a wall thickness between 0.03 mm and 0.2 mm.

Further in accordance with an embodiment of the present disclosure the pressure sensor is coated with a waterproof coating.

Still further in accordance with an embodiment of the present disclosure the waterproof coating is selected from a group consisting of Parylene, silicon, and polyurethane.

Additionally in accordance with an embodiment of the present disclosure, the apparatus includes the irrigation-aspiration sub-system, which is configured to convey irrigation fluid along the irrigation channel at a rate of between 1 and 5 milliliters per minute.

Moreover in accordance with an embodiment of the present disclosure, the apparatus includes the irrigation-aspiration sub-system configured to convey irrigation fluid along the irrigation channel intermittently providing periods of irrigation activity and intervening periods of irrigation inactivity, and a processor configured to sample respective values from the signal provided by the pressure sensor corresponding to time values during the periods of irrigation inactivity, and compute pressure values responsively to the respective sampled values.

Further in accordance with an embodiment of the present disclosure, the apparatus includes the irrigation-aspiration sub-system configured to convey irrigation fluid along the irrigation channel responsively to a modulated irrigation rate providing periods of irrigation activity above a given irrigation rate and intervening periods of irrigation activity below the given irrigation rate, and a processor configured to sample respective values from the signal provided by the pressure sensor corresponding to time values during the periods of irrigation activity below the given irrigation rate, and compute pressure values responsively to the respective sampled values.

Still further in accordance with an embodiment of the present disclosure, the apparatus includes a processor configured to compute a pressure value responsively to the signal provided by the pressure sensor, and control the irrigation-aspiration sub-system responsively to the computed pressure value.

There is also provided in accordance with still another embodiment of the present disclosure, an ophthalmic method, including providing an ophthalmic curette apparatus, including a handle having a distal end, a tube having a proximal end connected with the distal end of the handle, and having a distal tip configured to be inserted into an eye, an irrigation channel extending from the proximal end to the distal tip of the tube, and a pressure sensor including a sensing surface and disposed at the distal tip of the tube, wherein the pressure sensor is configured to be inserted into the eye and provide a signal responsively to intraocular pressure inside the eye, wherein the irrigation channel is configured to be coupled with an irrigation-aspiration sub-system to convey irrigation fluid along the irrigation channel, and wherein the irrigation channel is shaped, and positioned with respect to the pressure sensor, to direct a flow of the irrigation fluid over the sensing surface of the pressure sensor, sampling values from the signal provided by the pressure sensor, computing a pressure value responsively to the signal provided by the pressure sensor, and adjusting an aspiration rate or irrigation rate responsively to the computed pressure value.

Additionally in accordance with an embodiment of the present disclosure the adjusting includes adjusting an aspiration rate of eye fluid and waste matter from the eye via an aspiration line of a phacoemulsification probe.

Moreover, in accordance with an embodiment of the present disclosure the adjusting includes adjusting an irrigation rate of the irrigation fluid to the eye via an irrigation line of a phacoemulsification probe.

Further in accordance with an embodiment of the present disclosure the adjusting includes adjusting an irrigation rate of the irrigation fluid to the eye via the irrigation channel of the ophthalmic curette apparatus.

Still further in accordance with an embodiment of the present disclosure, the method includes conveying irrigation fluid along the irrigation channel intermittently providing periods of irrigation activity and intervening periods of irrigation inactivity, wherein the sampling includes sampling respective values from the signal provided by the pressure sensor corresponding to time values during the periods of irrigation inactivity, and the computing includes computing pressure values responsively to the respective sampled values.

Additionally in accordance with an embodiment of the present disclosure, the method includes conveying irrigation fluid along the irrigation channel responsively to a modulated irrigation rate providing periods of irrigation activity above a given irrigation rate and intervening periods of irrigation activity below the given irrigation rate, wherein the sampling includes sampling respective values from the signal provided by the pressure sensor corresponding to time values during the periods of irrigation activity below the given irrigation rate, and the computing includes computing pressure values responsively to the respective sampled values.

There is also provided in accordance with still another embodiment of the present disclosure, a method to manufacture an ophthalmic curette, the method including connecting a proximal end of a tube to a distal end of a handle, providing an irrigation channel extending from the proximal end to a distal tip of the tube, disposing a pressure sensor at the distal tip of the tube so that the pressure sensor provides a signal responsively to intraocular pressure inside the eye, and wherein the providing and the disposing are performed so that the irrigation channel is shaped, and positioned with respect to the pressure sensor, to direct a flow of irrigation fluid from the irrigation channel over a sensing surface of the pressure sensor.

Moreover, in accordance with an embodiment of the present disclosure the disposing includes disposing the pressure sensor at least partially in the irrigation channel.

Further in accordance with an embodiment of the present disclosure, the method includes disposing the irrigation channel in the tube and extending from the proximal end to a distal tip of the tube, the disposing the pressure sensor includes disposing the pressure sensor at least partially in the irrigation channel at the distal tip.

Still further in accordance with an embodiment of the present disclosure the tube has a beveled opening, which extends longitudinally along all of the distal tip of the tube.

Additionally in accordance with an embodiment of the present disclosure the beveled opening defines a plane which has an angle in the range of 30 to 70 degrees with a plane perpendicular to a direction of elongation of the tube.

Moreover, in accordance with an embodiment of the present disclosure the tube has a minimum length of 2 cm.

Further in accordance with an embodiment of the present disclosure the tube has an outer diameter between 0.1 mm and 0.8 mm.

Still further in accordance with an embodiment of the present disclosure the tube has a wall thickness between 0.03 mm and 0.2 mm.

Additionally in accordance with an embodiment of the present disclosure, the method includes coating the pressure sensor with a waterproof coating.

Moreover, in accordance with an embodiment of the present disclosure the waterproof coating is selected from a group consisting of Parylene, silicon, and polyurethane.

Further in accordance with an embodiment of the present disclosure, the method includes calibrating the pressure sensor after the coating.

### BRIEF DESCRIPTION OF THE DRAWINGS

The present invention will be understood from the following detailed description, taken in conjunction with the drawings in which:

FIG. 1 is a partly pictorial, partly block diagram view of an ophthalmic curette device constructed and operative in accordance with an embodiment of the present invention;

FIG. 2 is a schematic view of the device of FIG. 1 being inserted into an eye of a living subject;

FIG. 3 is a cross-sectional view of the device through line A:A of FIG. 1;

FIGS. 4 and 5 are views of the distal end of the device of FIG. 1;

FIG. 6 is a cutaway view of the distal end of the device of FIG. 1;

FIG. 7 is a schematic view of a distal end of an ophthalmic curette device constructed and operative in accordance with an alternative embodiment of the present invention;

FIG. 8 is a schematic view of a distal end of an ophthalmic curette device constructed and operative in accordance with yet another alternative embodiment of the present invention;

FIG. 9 is a cross-sectional view through line A:A of FIG. 8;

FIG. 10 is a schematic view of the distal end of the ophthalmic curette device of FIG. 8;

FIG. 11 is a schematic view of a pressure sensor feedback system using the ophthalmic curette device of FIG. 8;

FIG. 12 is a flowchart including steps in a method of operation of the system of FIG. 11; and

FIG. 13 is a flowchart including steps in a method of manufacture of the ophthalmic curette device of FIG. 8.

### DESCRIPTION OF EXAMPLE EMBODIMENTS

### Overview

During cataract surgery, a phacoemulsification probe is typically used with an ophthalmic curette to assist in removal of the cataract from the eye. The curette and the phacoemulsification probe require separate incisions into the eye. However, to make measurements on the eye during the surgery, such as measuring intraocular pressure, a different probe needs to be used.

One solution includes providing an ophthalmic curette which includes a handle and a tube (connected to the handle) for inserting through a cataract incision into a chamber of an eye of a living subject. A pressure sensor may be inside the tube, typically at its distal end, in order to take pressure readings in the chamber. The ophthalmic curette may therefore be used to manipulate cataract material during a procedure as well as provide pressure readings thereby removing the need for further tools to be used and/or further incisions. However, the ophthalmic curette suffers from a problem. During a phacoemulsification procedure, readings from the pressure sensor may become compromised because emulsified material adheres to the sensing surface of the pressure sensor.

Therefore, embodiments of the invention incorporate an irrigation channel (separate from an irrigation line used for the phacoemulsification probe) into the curette. The irrigation channel is shaped, and positioned with respect to the sensing surface of the pressure sensor, to be able to direct irrigation fluid over the sensing surface to remove emulsified material. The irrigation fluid may be conveyed at any suitable rate, e.g., 2 milliliters per second or in the range of 1 to 5 milliliters per minute.

In some embodiments, the irrigation fluid may be conveyed intermittently, or the irrigation rate may be modulated. In these embodiments, a signal supplied by the pressure sensor may be sampled when the irrigation fluid is not being conveyed or when the irrigation rate is below a threshold value. Sampling the signal when the irrigation fluid is not being conveyed or when the irrigation rate is below the threshold value may result in more accurate pressure measurements.

The pressure sensor is placed so that when the tube is inserted into the eye, the pressure sensor is also in the eye and exposed to the eye fluid. In some embodiments, the pressure sensor is coated with a waterproof coating such as Parylene, silicon, or polyurethane.

In some embodiments, the pressure sensor is at least partially placed in the irrigation channel, which extends through the tube to a distal tip of the tube. In some embodiments, the pressure sensor is at least partially placed in the distal tip.

In some embodiments, the tube may include a beveled opening, which extends longitudinally along all of the distal tip of the tube. The pressure sensor is at least partially placed in the distal tip. In this manner, the sensing surface of the pressure sensor is exposed directly to the fluid in the chamber of the eye while being cleaned by the flowing irrigation fluid from the irrigation channel. The beveled opening may have any suitable angle with the end of the tube. In some embodiments, the beveled opening defines a plane which has an angle in the range of 30 to 70 degrees with a plane perpendicular to a direction of elongation of the tube.

The tube may be made of any suitable material, for example, a polymer tube or a metal tube such as a rigid biocompatible metal tube. The tube may have any suitable cross-section shape for example, circular, elliptical, or rectangular. The tube may have any suitable form. In some embodiments, the tube is straight. In other embodiments, the tube is bent or curved. The tube may be formed from any suitable material, e.g., a polymer such as Polyether ether ketone (PEEK) or fluorinated ethylene propylene (FEP), or a metal such as stainless steel or titanium.

The tube may have any suitable length, which is long enough for the tube to be inserted into the eye and be maneuvered during the medical procedure. In some embodiments, the tube has a minimum length of 2 cm. The tube may have any suitable outer diameter and wall thickness. In some embodiments, the tube has an outer diameter between 0.1 mm and 0.8 mm, and a wall thickness between 0.03 mm and 0.2 mm.

In some embodiments, the device includes a processor and a display. In some embodiments, the processor is configured to compute a pressure value responsively to the signal provided by the pressure sensor. In an embodiment, the processor and the display may be disposed in the handle. In some embodiments, a display may also be disposed in a remote console, which is connected to the handle wirelessly and/or via wires, to also render the pressure value. The handle and the console may each include an interface to provide data communication between the handle and the console. In an embodiment, a processor may be disposed in the handle or the console. In other embodiments, the display and/or the processor are not included in the handle or the console.

In some embodiments, the sensor signal(s) and/or computed pressure values are conveyed via wires and/or wirelessly to a remote processing device which processes the sensor signals and/or uses the computed pressure values as part of a medical procedure process. For example, an irrigation rate and/or an aspiration rate of a phacoemulsification probe may be adjusted according to the pressure values.

### System Description

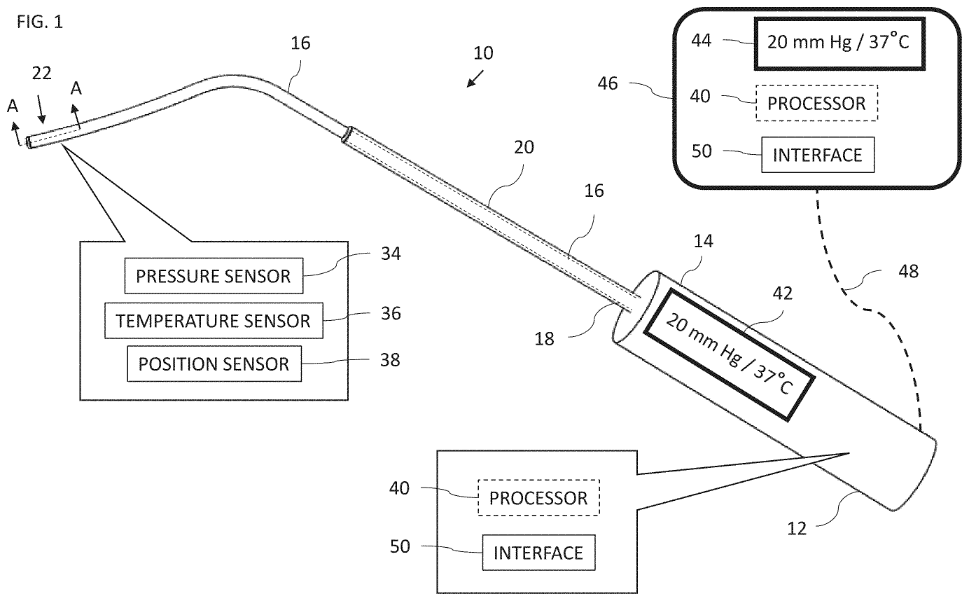

Reference is now made to FIG. 1, which is a partly pictorial, partly block diagram view of an ophthalmic curette device 10 constructed and operative in accordance with an embodiment of the present invention.

The ophthalmic curette device 10 includes a handle 12 having a distal end 14. The handle 12 may be formed from any suitable material or combination of materials, for example, a metal and/or polymer.

The ophthalmic curette device 10 includes a rigid biocompatible tube 16 having a proximal end 18 connected to the distal end 14 of the handle 12. The tube 16 may have any suitable form. In some embodiments, the tube 16 is straight. In other embodiments, the tube 16 is bent or curved to any suitable angle, as shown in FIG. 1. The tube 16 may be formed from any suitable metal, for example, stainless steel or titanium. In some embodiments, the tube 16 may be replaced by a tube of any suitable material, for example, a polymer such as PEEK or FEP. Suitable metal and polymer tubing are commercially available from IDEX corporation, Lake Forest, Ill., USA.

A proximal portion of the tube 16 is optionally surrounded by an elongated supporting member 20 to provide additional support to the proximal portion which is not inserted into an eye. The supporting member 20 may be formed from any suitable material, for example, a metal such as stainless steel or titanium, or a polymer. The tube 16 has a distal end 22.

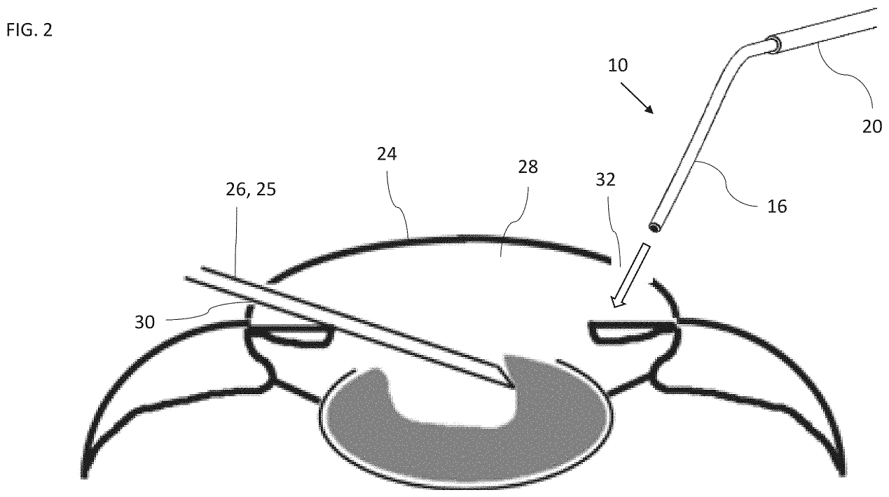

Reference is now made to FIG. 2, which is a schematic view of the device 10 of FIG. 1 being inserted into an eye 24 of a living subject.

FIG. 2 shows a needle 26 of a phacoemulsification probe 25 configured to be inserted into a chamber 28 of the eye 24 through an incision 30. A distal end of the tube 16 of the ophthalmic curette device 10 is configured to be inserted through a second incision 32 into the chamber 28 of the eye 24.

The tube 16 (which extends beyond the supporting member 20) may have any suitable length, which is long enough for the tube 16 to be inserted into the eye 24 and be maneuvered during the medical procedure. In some embodiments, the tube 16 (which extends beyond the supporting member 20) has a minimum length of 2 cm. The tube 16 may have any suitable outer diameter and wall thickness. In some embodiments, the tube 16 has an outer diameter between 0.1 mm and 0.8 mm, and a wall thickness between 0.03 mm and 0.2 mm.

Referring again to FIG. 1., in some embodiments, the ophthalmic curette device 10 includes a pressure sensor 34 disposed inside the tube 16 at the distal end 22 of the tube 16. The pressure sensor 34 is configured to provide a signal responsively to intraocular pressure inside the chamber 28 (FIG. 2) of the eye 24 (FIG. 2). Any suitable pressure sensor, which is small enough to insert inside the tube 16, and is sensitive enough to accurately measure the intraocular pressure, may be used. A suitable pressure sensor (e.g., Novasensor P330 series absolute pressure sensor die) is commercially available from Amphenol Thermometrics Inc., St. Mary's, Pa. 15857, United States. The Novasensor P330 series absolute pressure sensor die has high pressure sensitivity (standard pressure range 450 to 1050 mmHg) and has a 330×180 microns cross section. The longest dimension of the P330 pressure sensor may be aligned parallel to the axis of the tube 16.

In some embodiments, the ophthalmic curette device 10 includes a temperature sensor 36 disposed inside the tube 16 at the distal end 22 of the tube 16. The temperature sensor 36 is configured to provide a signal responsively to a temperature inside the chamber 28 (FIG. 2) of the eye 24 (FIG. 2). Suitable miniature medical temperature sensors are commercially available from ATC Semitec Ltd, Anderton, Northwich, Cheshire, CW9 6FY, U.K.

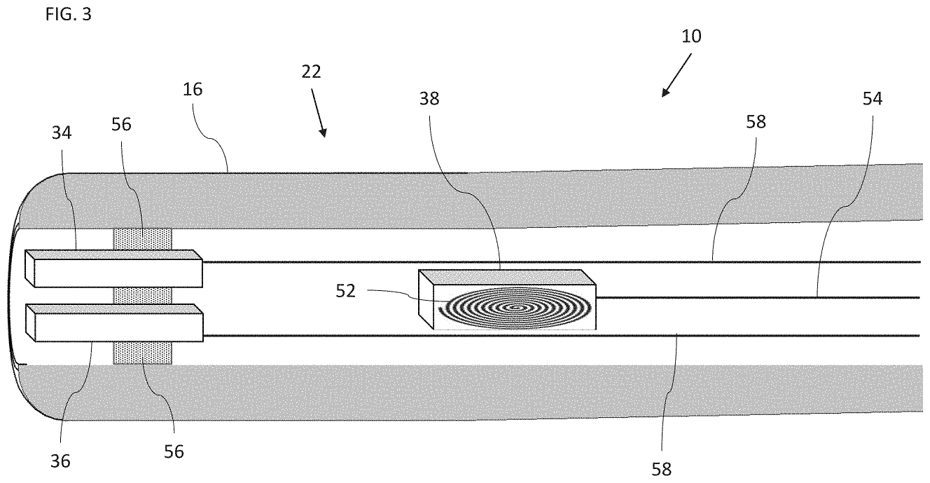

In some embodiments, the ophthalmic curette device 10 includes the pressure sensor 34 and the temperature sensor 36. The pressure sensor 34 and the temperature sensor 36 are described in more detail with reference to FIG. 3.

In some embodiments, the ophthalmic curette device 10 includes a magnetic position sensor 38 disposed inside the tube 16. The magnetic position sensor 38 may be configured to be used as part of a position tracking system (not shown) for tracking a position of the distal end 22 of the tube 16. As the tube 16 is rigid, the magnetic position sensor 38 may be disposed at any suitable position inside the tube 16. The magnetic position sensor 38 is described in more detail with reference to FIG. 3.

The ophthalmic curette device 10 optionally includes a processor 40 and a display 42. In some embodiments, the processor 40 is configured to compute a pressure value responsively to the signal provided by the pressure sensor 34. In some embodiments, the processor 40 is configured to compute a temperature value responsively to the signal provided by the temperature sensor 36. The display 42 is configured to render the pressure value and/or the temperature value.

The processor 40 and the display 42 may be disposed in/on the handle 12. The display 42 may include any suitable display, for example, a liquid crystal display.

In some embodiments, a display 44 may also be disposed in a remote console 46, which is connected with the handle 12 wirelessly and/or via wires 48. The display 44 is configured to render the pressure value and/or the temperature value. The handle 12 and the console 46 may each include an interface 50 to provide data communication between the handle 12 and the console 46. The interfaces 50 may comprise circuitry to allow wired and/or wireless communication therebetween.

In some embodiments, the display 44 may be disposed in the remote console 46 without a display on the handle 12. In these embodiments, the processor 40 may be disposed in the handle 12 or the console 46.

In practice, some or all of the functions of the processor 40 may be combined in a single physical component or, alternatively, implemented using multiple physical components. These physical components may comprise hard-wired or programmable devices, or a combination of the two. In some embodiments, at least some of the functions of the processor 40 may be carried out by a programmable processor under the control of suitable software. This software may be downloaded to a device in electronic form, over a network, for example. Alternatively, or additionally, the software may be stored in tangible, non-transitory computer-readable storage media, such as optical, magnetic, or electronic memory.

In other embodiments, the displays 42, 44 and/or the processor 40 are not included in the handle 12 or the console 46.

In some embodiments, the sensor signal(s) and/or computed temperature and/or pressure values are conveyed via wires and/or wirelessly to a remote processing device (not shown) which processes the sensor signals and/or uses the computed temperature and/or pressure values as part of a medical procedure process. For example, medical procedure parameters such as phacoemulsification probe needle vibration frequency, amplitude, and/or mode may be adjusted according to the temperature and/or pressure values.

Reference is now made to FIG. 3, which is a cross-sectional view of the ophthalmic curette device 10 through line A:A of FIG. 1.

The magnetic position sensor 38 may include at least one coil 52. The magnetic position sensor 38 may be implemented as a single axis sensor (SAS) with one coil, as a dual axis sensor (DAS) with two orthogonally disposed coils, or a triple axis sensor (TAS) with three orthogonally disposed coils. Each coil may be a printed coil or a wound coil, e.g., wound on a suitable magnetic core. The ophthalmic curette device 10 may include a cable 54 connecting the magnetic position sensor 38 with the processor 40 (FIG. 1) or the interface 50 (FIG. 1) in the handle 12 (FIG. 1).

Magnetic field generators may be placed at known locations external to the patient, for example, around the head of the patient or in a collar around the neck of the patient. The magnetic position sensor 38 generates electrical signals in response to these magnetic fields, which may be processed to determine a position (e.g., location and/or orientation) of the distal end 22 of the ophthalmic curette device 10. Magnetic tracking system are described in U.S. Pat. Nos. 5,391,199, 6,690,963, 6,484,118, 6,239,724, 6,618,612 and 6,332,089, in PCT International Publication No. WO 1996/005768, and in U.S. Patent Application Publications Nos. 2002/0065455 and 2003/0120150 and 2004/0068178. The magnetic position sensor 38 may allow the ophthalmic curette device 10 to be tracked during use, for example, during robotic guided surgery in which the ophthalmic curette device 10 may be robotically guided.

In some embodiments, the coil may include a magnetic core. For example, magnetic antennas can use a magnetic core made of a ferromagnetic or ferrimagnetic material such as iron or a nickel-zinc ferrite or magnesium-zinc ferrite to increase permeability. A magnetic core can increase the sensitivity of an antenna by a factor of up to several thousand, by increasing the magnetic field due to its higher magnetic permeability. Therefore, coils used in navigable probes typically include coils with a magnetic core. A solid magnetic core may be constructed by any suitable method including joining magnetic-core powder using a binder and/or very high temperatures (sintering) to form a solid mass. In some embodiments, the coil may include a magnetic core produced by one of the above methods. However, the above production methods are generally not suitable for producing solid magnetic cores which are small enough to insert into a coil having an inner diameter of about 500 microns or less.

Therefore, in some embodiments, a magnetic core may be formed from a tube containing separate powder granules of a ferrite. The tube may have any suitable inner diameter. The inner diameter of the tube may be in the range of 100 to 750 microns. A coil is then placed around the tube, for example, by inserting the tube into the coil. The coil may be covered with a covering keeping the coil in place and acting as a biocompatible cover, for example, but not limited to a plastic cover such as a plastic tube, or with a coating such an as enamel or epoxy paint, shrink sleeve, or metal cover. A metal cover may also provide shielding from high frequency electromagnetic interference. The wire (e.g., copper wire) used in the coil may have any suitable gauge, for example, but not limited to 60 gauge which is about 8 microns in diameter. The powder granules are held in place by the tube. The powder granules may be bound together using a binder material such as epoxy. The tube may be formed from any suitable material such as a wide range of thermoplastics, e.g., polyimide, polyamide, polyethylene terephthalate (PET), fluorinated ethylene propylene (FEP), or polyvinyl chloride (PVC) or other materials such as an engineered ceramic, a carbon material, or a non-ferromagnetic metal. The tube provides a controlled outer diameter surface on which to slide the coil.

As the powder granules may have a size of about 40 microns, it may be difficult to place the powder granules into the tube. Therefore, in some embodiments, separate powder granules of a ferrite are introduced into the tube using the following method. The tube may be made of any suitable material which can be heat-shrunk, for example, but not limited to, a wide-range of thermoplastics, e.g., polyamide, polyethylene terephthalate (PET), fluorinated ethylene propylene (FEP), or polyvinyl chloride (PVC). While the powder granules are introduced into the tube, the tube typically has an outer diameter which is greater than an inner diameter of the coil. For example, if the inner diameter of the coil is 180 microns, the outer diameter of the tube into which the powder granules are disposed has an outer diameter of 250 microns and an inner diameter of about 180 microns. The outer diameter of the tube shrinks to 180 microns after being heat shrunk. The preshrunk tube may have any suitable outer diameter in accordance with the inner diameter of the coil and the heat shrink properties of the plastic tube. The preshrunk tube may have any suitable outer diameter, for example, in the range of 150-1200 microns. The powder granules may be bound together after being placed in the tube using a binder such as a low viscosity epoxy, which may be wicked into the tube with capillary action after the tube is shrunk, or before the tube is shrunk and then the tube is shrunk while the epoxy is still liquid as it would not be possible to shrink the tube once the epoxy is cured. The powder granules may have any suitable size which is less than the inner diameter of the tube into which the powder granules are disposed. The powder granules may be introduced into the tube using any suitable method. For example, the powder granules may be introduced into the tube with the aid of a funnel which is aligned with the tube opening. The funnel is filled with the powder granules and is vibrated up and down, and/or side-to-side, using any suitable vibration method, such as an ultrasonic method. The funnel may be connected with the tube, and vibrated in unison with the tube to facilitate the introduction of the powder granules into the tube. Additionally, or alternatively, one or more magnets (electromagnetics and/or permanent magnets) may be connected to the tube to facilitate introduction of the powder granules into the tube. For example, using a magnet connected to the bottom of the tube and/or a ring-type magnet connected around the tube. The magnet(s) may allow movement (up and down, and/or sideways movement), vibration (up and down, and/or sideways vibration), and/or rotation of the tube.

In some embodiments, the powder granules may be introduced into a tube without subsequently heat shrinking the tube. In these embodiments, the powder granules are suspended in a liquid, such as an alcohol, for example, but not limited to, isopropyl alcohol, or any other liquid which has a sufficiently low viscosity and a high enough evaporation rate. The suspending may be performed using any suitable method for example, but not limited to, placing the powder granules with the liquid in a container on a vibration table or by using any other vibration method, such as using ultrasound. The percentage of powder granules in the suspension, by volume, may be any suitable value, for example, in the range of 30-70%. An end of an empty tube is placed in the liquid so that capillary action draws some of the liquid with the powder granules into the tube. The tube has an outer diameter less than the inner diameter of the coil, for example, in the range of 150 to 800 microns. The inner diameter of the tube is generally in the range of 100 to 750 microns. The tube may be made of any suitable material, for example, but not limited to, plastic, an engineered ceramic, a carbon material, or a non-ferromagnetic metal. Once the liquid-granule mixture is in the tube, the liquid is evaporated from the tube using any suitable method such as using heat and/or by blowing air over the tube. After the evaporation, a binder such as a low viscosity epoxy may be wicked into the tube with capillary action to bind the powder granules together.

The pressure sensor 34 may be disposed at the distal tip of the tube 16. In some embodiments, the temperature sensor 36 may be disposed next to the pressure sensor 34 at the distal tip if there is enough room in the distal tip for both sensors 34, 36. The pressure sensor 34 and the temperature sensor 36 may be connected to the inner surface of the tube 16 using a suitable adhesive 56, which also prevents liquid from reaching electrical connections, wires and the magnetic position sensor 38.

If there is not enough room for both sensors 34, 36 at the distal tip, the pressure sensor 34 is generally disposed at the distal tip and the temperature sensor 36 is disposed proximally to the pressure sensor 34, or vice-versa. The magnetic position sensor 38 is generally, but not necessarily, disposed proximally to the pressure sensor 34 and the temperature sensor 36.

The pressure sensor 34 and the temperature sensor 36 may be connected using wires/cables 58 to the processor 40 (FIG. 1) and/or the interface 50 (FIG. 1) in the handle 12 (FIG. 1). In some embodiments, the wires/cables 58 may extend out of the handle 12 for connection to the remote console 46 (FIG. 1).

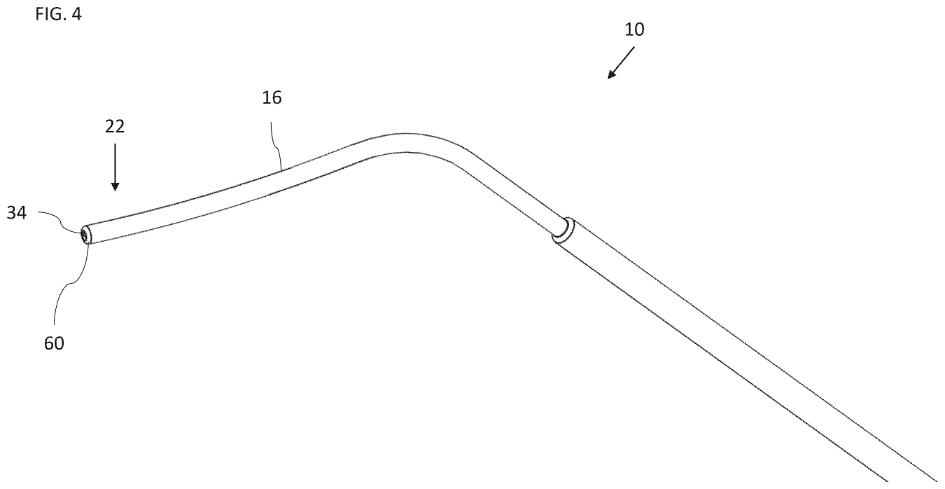

Reference is now made to FIGS. 4 and 5, which are views of the distal end 22 of the ophthalmic curette device 10 of FIG. 1. FIG. 4 shows that the distal end 22 has a smooth or rounded edge 60. FIG. 5 shows the pressure sensor 34 disposed in the distal end 22 of the tube 16. The temperature sensor 36 and the magnetic position sensor 38 are not shown in FIGS. 4 and 5 for the sake of simplicity.

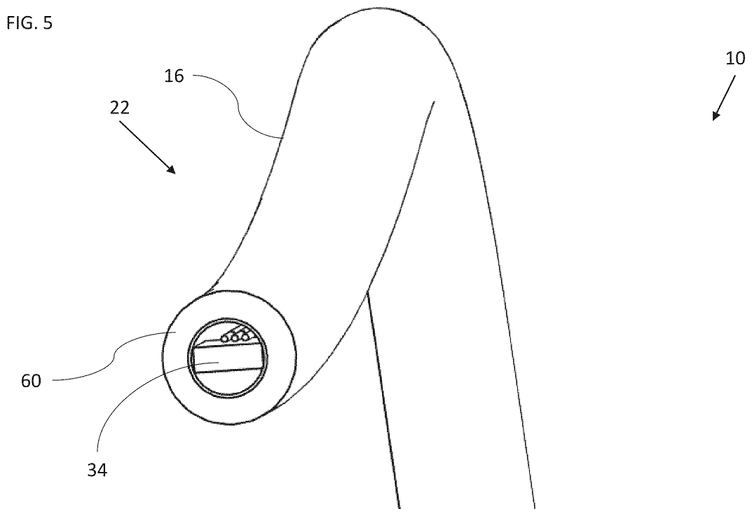

Reference is now made to FIG. 6, which is a cutaway view of the distal end of the ophthalmic curette device 10 of FIG. 1. FIG. 6 shows the cables 58 extending from the pressure sensor 34 down the inside of the tube 16 towards the handle 12 (FIG. 1). FIG. 6 also illustrates the supporting member 20 surrounding the proximal portion of the tube 16. The temperature sensor 36, the magnetic position sensor 38, and their associated wires/cables are not shown in FIG. 6 for the sake of simplicity.

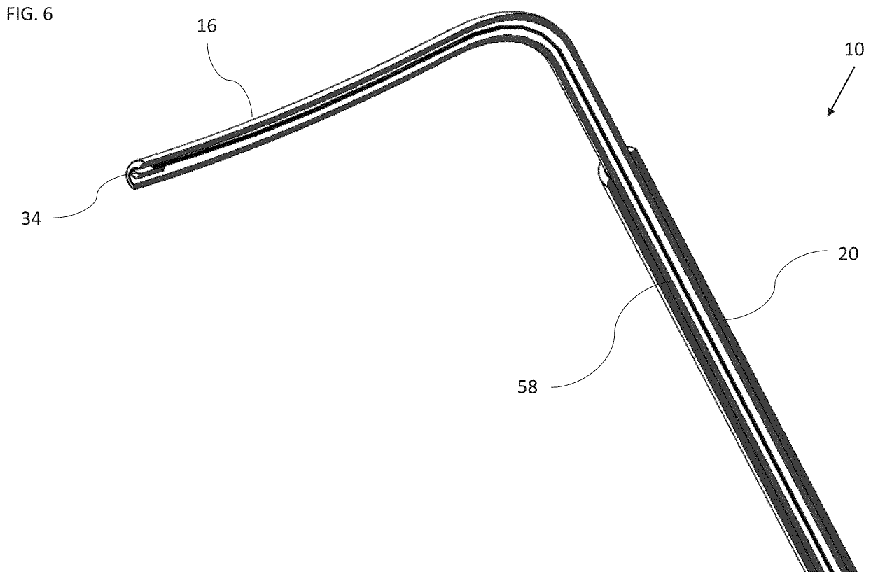

Reference is now made to FIG. 7, which is a schematic view of a distal end of an ophthalmic curette device 62 constructed and operative in accordance with an alternative embodiment of the present invention. The ophthalmic curette device 62 is substantially the same as the ophthalmic curette device 10 except that the ophthalmic curette device 62 includes a loop 64 or hook connected to the distal end 22 of the tube 16. The width of the loop is typically about the size of the outer diameter of the tube 16. The length of the loop 64 extending away from the distal end 22 may have any suitable size, for example, between half and twice the size of the outer diameter of the tube 16.

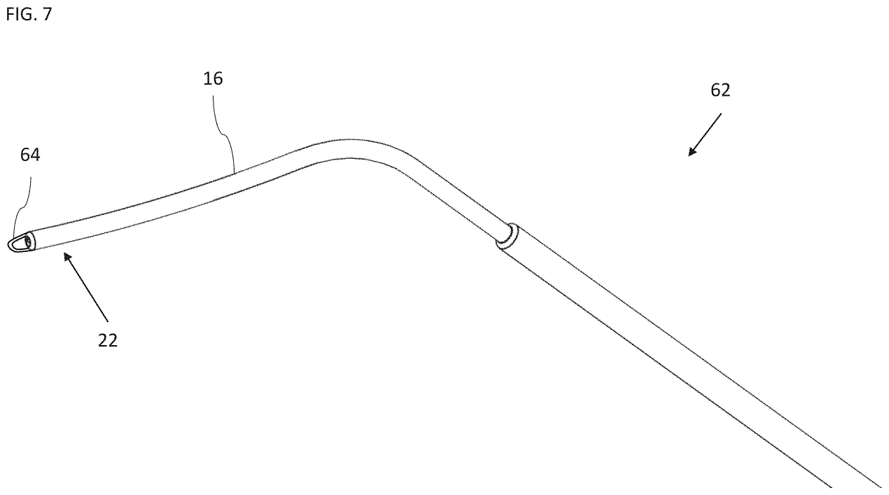

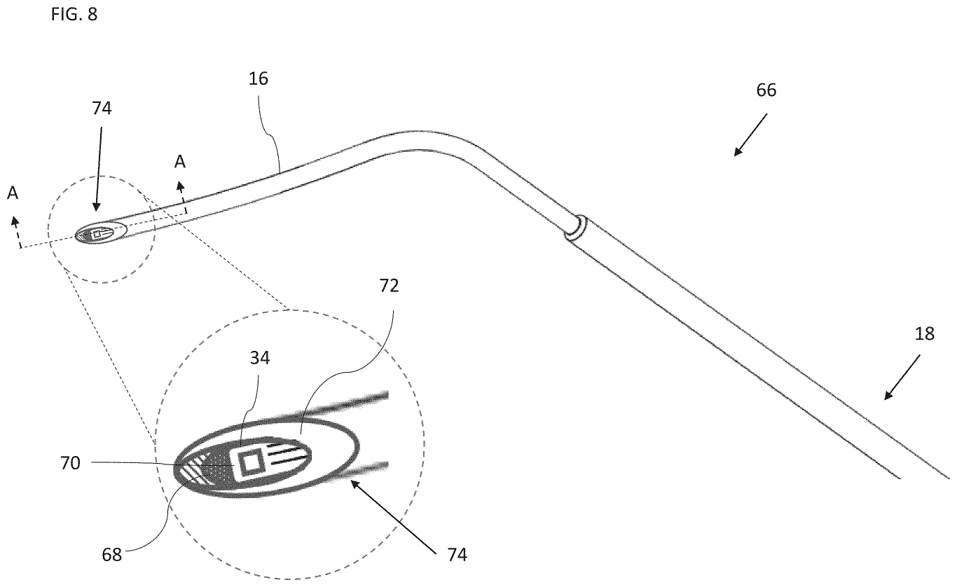

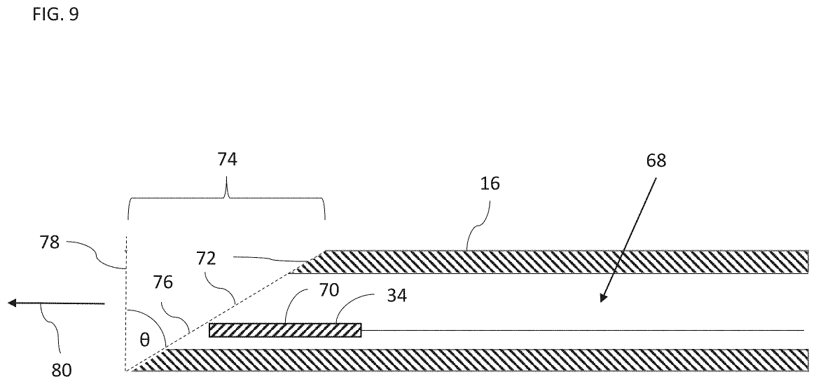

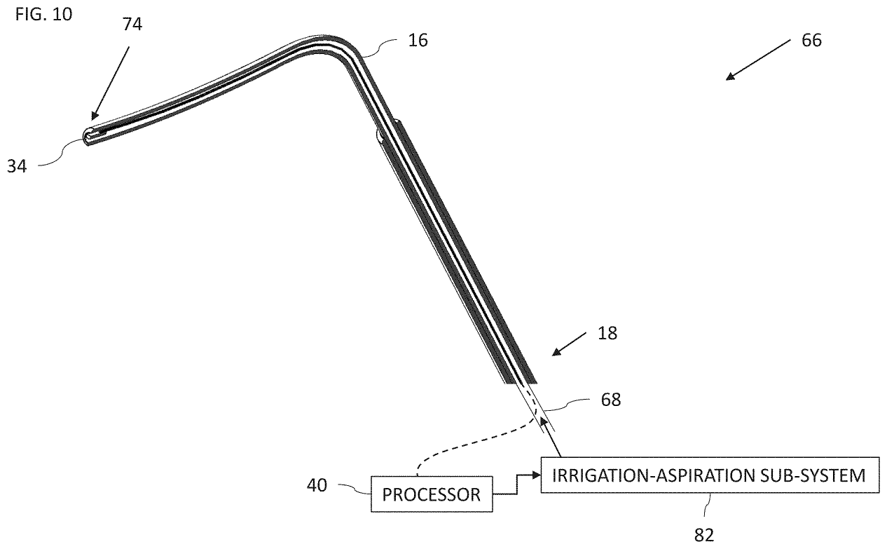

Reference is now made to FIG. 8, which is a schematic view of a distal end of an ophthalmic curette device 66 constructed and operative in accordance with yet another alternative embodiment of the present invention. The ophthalmic curette device 66 is substantially the same as the ophthalmic curette device 10 except that the ophthalmic curette device 66 includes an irrigation channel 68 extending from the proximal end 18 to a distal tip 74 of the tube 16. The extent of the irrigation channel 68 is shown in more detail with reference to FIGS. 9 and 10. The pressure sensor 34 is disposed at the distal tip 74 of the tube 16, and configured to be inserted into the chamber 28 of the eye 24 (FIGS. 2 and 11) and provide a signal responsively to intraocular pressure inside the chamber 28 of the eye 24. The pressure sensor 34 includes a sensing surface 70 (FIG. 9). In some embodiments, the lumen of the tube 16 provides the walls of the irrigation channel 68. In some embodiments, a separate irrigation channel 68 with its own walls is disposed in lumen of the tube 16. In some embodiments, tube 16 may include multiple lumens, one for the irrigation channel 68 and one to hold wires of the pressure sensor 34. In some embodiment, the irrigation channel 68 may be disposed externally to the tube 16.

The irrigation channel 68 is shaped, and positioned with respect to the pressure sensor 34, to direct a flow of irrigation fluid over the sensing surface 70 of the pressure sensor 34. In some embodiments, the pressure sensor 34 is at least partially disposed in the irrigation channel 68, as described in more detail with reference to FIG. 9. In some embodiments, the distal tip 74 of the tube 16 includes a beveled opening 72, in which the pressure sensor 34 is at least partially disposed. The beveled opening 72 and the positioning of the pressure sensor 34 are described in more detail with reference to FIG. 9.

Any suitable pressure sensor, which is small enough to insert inside the tube 16, and is sensitive enough to accurately measure the intraocular pressure, may be used. A suitable pressure sensor (e.g., Novasensor P330 series absolute pressure sensor die) is commercially available from Amphenol Thermometrics Inc., St. Mary's, Pa. 15857, United States. The Novasensor P330 series absolute pressure sensor die has high pressure sensitivity (standard pressure range 450 to 1050 mmHg) and has a 330×180 microns cross section. The longest dimension of the P330 pressure sensor may be aligned parallel to the axis of the tube 16. In some embodiments, the pressure sensor 34 is coated with a waterproof coating. Any suitable waterproof coating may be used, for example, Parylene, silicon, or polyurethane. As the coating may change the pressure sensing characteristics of the pressure sensor 34, the signal provided by the pressure sensor 34 is generally recalibrated after applying the coating and/or after installing the coated pressure sensor 34 in the tube 16.

Reference is now made to FIG. 9, which is a cross-sectional view through line A:A of FIG. 8. FIG. 9 shows the pressure sensor 34 disposed in the irrigation channel 68, which is in the tube 16 and formed by the inner walls of the tube 16. In some embodiments, the pressure sensor 34 is at least partially disposed in the irrigation channel 68 (i.e., the pressure sensor 34 may be disposed at least partially externally to the irrigation channel 68 and tube 16). The tube 16 includes a distal tip 74.

In some embodiments, the irrigation channel 68 is disposed in the tube 16 and extends from the proximal end 18 of the tube 16 to the distal tip 74 (as shown in FIG. 10), and the pressure sensor 34 is disposed at least partially in the irrigation channel 68 at the distal tip 74.

In some embodiments, the tube 16 includes the beveled opening 72 (including a beveled cut of the end of the distal tip 74 of the tube 16), which extends longitudinally along all of the distal tip 74 of the tube 16 (i.e., the extent of the beveled opening 72 including the beveled cut on the end of the tube 16 defines the longitudinal extent of the distal tip 74). The angle of the beveled opening 72 may be any suitable angle which allows the pressure sensor 34 to be disposed in the irrigation channel 68 while exposing the sensing surface 70 directly to the eye fluid while also allowing the irrigation fluid to clean the sensing surface 70 of debris. In some embodiments, the beveled opening 72 defines a plane 76 which has an angle (θ) in the range of 30 to 70 degrees with a plane 78 perpendicular to a direction of elongation 80 of the tube 16. The beveled opening 72 allows more surface area of the pressure sensor 34 to be exposed to the fluid in the eye 24 while still providing protection for the pressure sensor 34.

Reference is now made to FIG. 10, which is a schematic view of the distal end of the ophthalmic curette device 66 of FIG. 8. FIG. 10 shows the irrigation channel 68 extending from the distal tip 74 of the tube 16 to the proximal end 18 of the tube 16.

As shown in FIG. 11, an irrigation-aspiration sub-system 82 is coupled with the irrigation channel 68 and is configured to convey irrigation fluid along the irrigation channel 68. In some embodiments, the irrigation-aspiration sub-system 82 may include one or more pumps. In other embodiments, the irrigation-aspiration sub-system 82 may include an irrigation bag which is raised above the working height of the ophthalmic curette device 66 to provide the irrigation fluid using a gravity-based feed.

The irrigation rate may be set to any suitable value. In some embodiments, irrigation-aspiration sub-system 82 is configured to convey the irrigation fluid along the irrigation channel 68 at a rate of between 1 and 5 milliliters per minute (for example, 2 milliliters per minute). In some embodiments, the irrigation-aspiration sub-system 82 is configured to convey the irrigation fluid at a constant rate throughout the use of the ophthalmic curette device 66 in the eye 24.

In other embodiments, irrigation-aspiration sub-system 82 is configured to convey irrigation fluid along the irrigation channel 68 intermittently providing periods of irrigation activity and intervening periods of irrigation inactivity. The processor 40 is configured to: sample respective values from the signal provided by the pressure sensor 34 corresponding to time values during the periods of irrigation inactivity; and compute pressure values responsively to the respective sampled values.

In yet other embodiments, the irrigation-aspiration sub-system 82 is configured to convey irrigation fluid along the irrigation channel 68 responsively to a modulated irrigation rate (e.g., according to a sine wave or triangular wave pattern) providing periods of irrigation activity above a given irrigation rate and intervening periods of irrigation activity below the given irrigation rate. The processor 40 is configured to: sample respective values from the signal provided by the pressure sensor 34 corresponding to time values during the periods of irrigation activity below the given irrigation rate; and compute pressure values responsively to the respective sampled values.

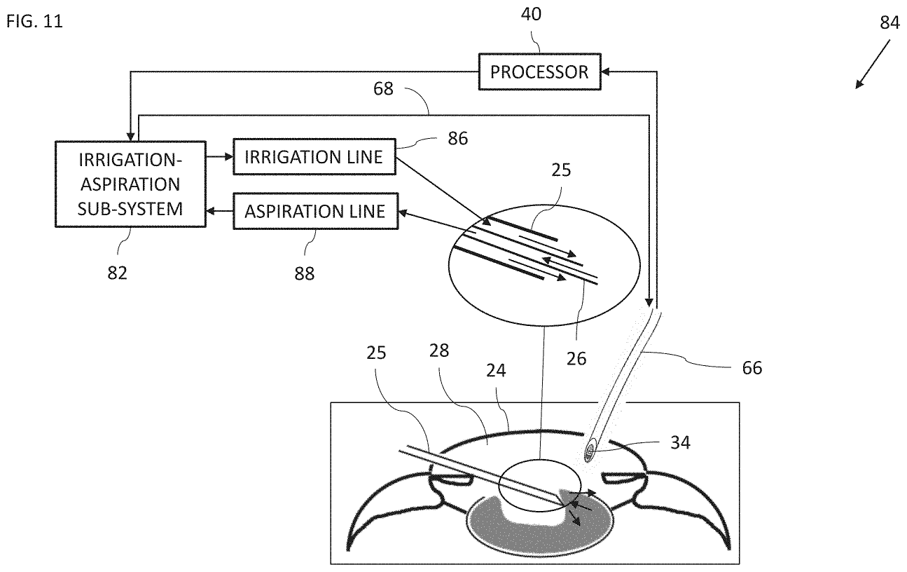

Reference is now made to FIGS. 11 and 12. FIG. 11 is a schematic view of a pressure sensor feedback system 84 using the ophthalmic curette device 66 of FIG. 8. FIG. 12 is a flowchart 100 including steps in a method of operation of the system 84 of FIG. 11.

The phacoemulsification probe 25 includes an irrigation line 86 and an aspiration line 88. The irrigation-aspiration sub-system 82 is coupled with the irrigation line 86 and the aspiration line 88. In some embodiments, the aspiration line 88 may be disposed in the needle 26 of the phacoemulsification probe 25 and the irrigation line 86 may be disposed around the needle 26. The irrigation-aspiration sub-system 82 is configured to convey irrigation fluid along the irrigation line 86 into the chamber 28 of the eye 24 and aspirate eye fluid and waste matter from the chamber 28 of the eye 24 via the aspiration line 88.

The processor 40 is configured to sample values from the signal provided by the pressure sensor 34 (block 102). The values may be sampled periodically, for example, in a range of every 5 milliseconds to every 100 milliseconds. The processor 40 is configured to compute a pressure value or values responsively to the signal provided by the pressure sensor 34 (block 104). The processor 40 is operationally connected to the irrigation-aspiration sub-system 82 using a wired and/or wireless connection.

The processor 40 is configured to control the irrigation-aspiration sub-system 82 to adjust at least one of the following responsively to the computed pressure value(s) (block 106): (a) an aspiration rate of eye fluid and waste matter from the eye 24 via the aspiration line 88 of the phacoemulsification probe 25; (b) an irrigation rate of the irrigation fluid to the eye 24 via the irrigation line 86 of the phacoemulsification probe 25; and/or (c) an irrigation rate of the irrigation fluid to the eye 24 via the irrigation channel of the ophthalmic curette device 66. For example, if the phacoemulsification probe 25 is removed from the eye 24 and the pressure sensor 34 indicates that the pressure of the fluid in the eye is too low, the irrigation rate of the irrigation fluid to the eye 24 via the irrigation channel 68 of the ophthalmic curette device 66 may be increased. By way of another example, if there is a sharp pressure drop in the eye 24, the irrigation-aspiration sub-system 82 may be configured to reduce or eliminate aspiration of eye fluid and waster matter via the aspiration line 88 and/or increase the irrigation rate of the irrigation fluid to the eye 24 via the irrigation channel 68 of the ophthalmic curette device 66 and/or increase the rate of irrigation fluid to the eye 24 via the irrigation line 86 of the phacoemulsification probe 25.

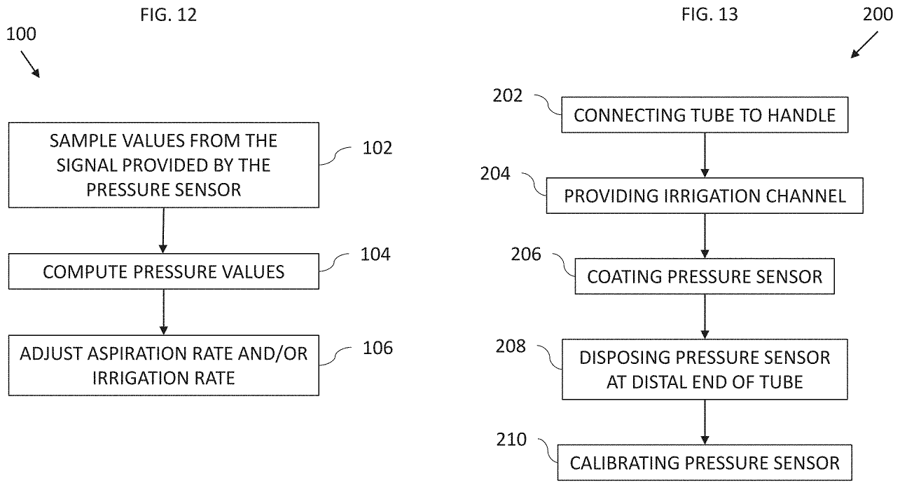

Reference is now made to FIG. 13, which is a flowchart 200 including steps in a method of manufacture of the ophthalmic curette device 66 of FIG. 8. Reference is also made to FIG. 8.

The method includes connecting the proximal end 18 of the tube 16 to the distal end 14 of the handle 12 (FIG. 1 and block 202). The method also includes providing the irrigation channel 68 extending from the proximal end 18 to the distal tip 74 of the tube 16 (block 204). The irrigation channel 68 may be provided by the inner channel of the tube 16. Therefore, in some embodiments, the method includes disposing the irrigation channel 68 in the tube 16 with the irrigation channel 68 extending from the proximal end 18 to the distal tip 74 of the tube 16.

In some embodiments, the step of block 202 includes providing the tube 16 with the beveled opening 72 or a making the beveled opening 72 in the tube 16. The beveled opening 72 (including the beveled cut) extends longitudinally along all of the distal tip 74 of the tube 16. The angle of the beveled opening 72 may be any suitable angle which allows the pressure sensor 34 to be disposed in the irrigation channel 68 while exposing the sensing surface 70 directly to the eye fluid while also allowing the irrigation fluid to clean the sensing surface 70 of debris. In some embodiments, the beveled opening 72 defines a plane 76 which has an angle (θ) in the range of 30 to 70 degrees with a plane 78 perpendicular to a direction of elongation 80 of the tube 16 (FIG. 9).

In some embodiments, such as when the pressure sensor 34 is not waterproof with respect to the irrigation fluid of the irrigation channel 68, the method also includes coating the pressure sensor 34 with any suitable waterproof coating (block 206), such as Parylene, silicon, and/or polyurethane.

The method also includes disposing the pressure sensor 34 at the distal tip 74 of the tube 22 so that the pressure sensor 34 provides a signal responsively to intraocular pressure inside the eye 24 (i.e., when the distal tip 74 is disposed in the eye 24) (block 208). The steps of blocks 204 and 208 are performed so that the irrigation channel 68 is shaped, and positioned with respect to the pressure sensor 34, to direct a flow of irrigation fluid from the irrigation channel 68 over the sensing surface 70 of the pressure sensor 34. In some embodiments, the step of block 208 includes disposing the pressure sensor 34 at least partially in the irrigation channel 68 optionally at the distal tip 74.

As coating the pressure sensor 34 may change the pressure sensing characteristics of the pressure sensor 34, the method optionally includes calibrating the signal provided by the pressure sensor 34 after applying the coating and/or after installing the coated pressure sensor 34 in the tube 16 (block 210). The calibration may be performed by sampling the signal provided by the pressure sensor 34 at known pressures in the expected range of pressures in the eye 24 against corresponding pressure readings from an accurate pressure sensor which is also waterproof (e.g., from an accurate pressure probe).

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 72% to 108%.

Various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination.

The embodiments described above are cited by way of example, and the present invention is not limited by what has been particularly shown and described hereinabove. Rather the scope of the invention includes both combinations and subcombinations of the various features described hereinabove, as well as variations and modifications thereof which would occur to persons skilled in the art upon reading the foregoing description and which are not disclosed in the prior art.

What is claimed is:

1. A phacoemulsification system, comprising:
a phacoemulsification probe configured to be inserted into an eye;
an ophthalmic curette, comprising:
a handle having a distal end;
a tube having a proximal end connected with the distal end of the handle, and having a distal tip configured to be inserted into the eye;
an irrigation channel extending from the proximal end to the distal tip of the tube; and
a pressure sensor comprising a sensing surface and disposed at the distal tip of the tube, wherein the pressure sensor is configured to be inserted into the eye and provide a signal responsively to intraocular pressure inside the eye; and
an irrigation-aspiration sub-system coupled with the irrigation channel and configured to convey irrigation fluid along the irrigation channel, and wherein the irrigation channel is shaped, and positioned with respect to the pressure sensor, to direct a flow of the irrigation fluid over the sensing surface of the pressure sensor.

2. The system according to claim 1, wherein the tube has a lumen, the irrigation channel having walls defined by the lumen.

3. The system according to claim 1, wherein the tube has a lumen, the irrigation channel having walls disposed in the lumen.

4. The system according to claim 1, wherein the pressure sensor is at least partially disposed in the irrigation channel.

5. The system according to claim 1, wherein the irrigation channel is disposed in the tube and extends from the proximal end to the distal tip, and wherein the pressure sensor is disposed at least partially in the irrigation channel at the distal tip.

6. The system according to claim 5, wherein the tube comprises a beveled opening, which extends longitudinally along all of the distal tip of the tube.

7. The system according to claim 6, wherein the beveled opening defines a plane which has an angle in the range of 30 to 70 degrees with a plane perpendicular to a direction of elongation of the tube.

8. The system according to claim 1, wherein the tube has a minimum length of 2 cm.

9. The system according to claim 1, wherein the tube has an outer diameter between 0.1 mm and 0.8 mm.

10. The system according to claim 9, wherein the tube has a wall thickness between 0.03 mm and 0.2 mm.

11. The system according to claim 1, wherein the pressure sensor is coated with a waterproof coating.

12. The system according to claim 11, wherein the waterproof coating is selected from a group consisting of Parylene, silicon, and polyurethane.

13. The system according to claim 1, wherein the irrigation-aspiration sub-system is configured to convey irrigation fluid along the irrigation channel at a rate of between 1 and 5 milliliters per minute.

14. The system according to claim 1, wherein the irrigation-aspiration sub-system is configured to convey irrigation fluid along the irrigation channel intermittently providing periods of irrigation activity and intervening periods of irrigation inactivity, the system further comprising a processor configured to: sample respective values from the signal provided by the pressure sensor corresponding to time values during the periods of irrigation inactivity; and compute pressure values responsively to the respective sampled values.

15. The system according to claim 1, wherein the irrigation-aspiration sub-system is configured to convey irrigation fluid along the irrigation channel responsively to a modulated irrigation rate providing periods of irrigation activity above a given irrigation rate and intervening periods of irrigation activity below the given irrigation rate, the system further comprising a processor configured to: sample respective values from the signal provided by the pressure sensor corresponding to time values during the periods of irrigation activity below the given irrigation rate; and compute pressure values responsively to the respective sampled values.

16. The system according to claim 1, further comprising a processor configured to compute a pressure value responsively to the signal provided by the pressure sensor, and wherein:
the phacoemulsification probe comprises an irrigation line and an aspiration line;
the irrigation-aspiration sub-system is coupled with the irrigation line and the aspiration line; and
the processor is configured to control the irrigation-aspiration sub-system to adjust at least one of the following responsively to the computed pressure value:
an aspiration rate of eye fluid from the eye via the aspiration line of the phacoemulsification probe;
an irrigation rate of the irrigation fluid to the eye via the irrigation line of the phacoemulsification probe; and
an irrigation rate of the irrigation fluid to the eye via the irrigation channel of the ophthalmic curette.

17. An ophthalmic curette apparatus, comprising:
a handle having a distal end;
a tube having a proximal end connected with the distal end of the handle, and having a distal tip configured to be inserted into an eye;
an irrigation channel extending from the proximal end to the distal tip of the tube; and
a pressure sensor comprising a sensing surface and disposed at the distal tip of the tube, wherein the pressure sensor is configured to be inserted into the eye and provide a signal responsively to intraocular pressure inside the eye, wherein the irrigation channel is configured to be coupled with an irrigation-aspiration sub-system to convey irrigation fluid along the irrigation channel, and wherein the irrigation channel is shaped, and positioned with respect to the pressure sensor, to direct a flow of the irrigation fluid over the sensing surface of the pressure sensor.

18. The apparatus according to claim 17, wherein the tube has a lumen, the irrigation channel having walls defined by the lumen.

19. The apparatus according to claim 17, wherein the tube has a lumen, the irrigation channel having walls disposed in the lumen.

20. The apparatus according to claim 17, wherein the pressure sensor is at least partially disposed in the irrigation channel.

21. The apparatus according to claim 17, wherein the irrigation channel is disposed in the tube and extends from the proximal end to the distal tip, and wherein the pressure sensor is disposed at least partially in the irrigation channel at the distal tip.

22. The apparatus according to claim 21, wherein the tube comprises a beveled opening, which extends longitudinally along all of the distal tip of the tube.

23. The apparatus according to claim 22, wherein the beveled opening defines a plane which has an angle in the range of 30 to 70 degrees with a plane perpendicular to a direction of elongation of the tube.

24. The apparatus according to claim 17, wherein the tube has a minimum length of 2 cm.

25. The apparatus according to claim 17, wherein the tube has an outer diameter between 0.1 mm and 0.8 mm.

26. The apparatus according to claim 25, wherein the tube has a wall thickness between 0.03 mm and 0.2 mm.

27. The apparatus according to claim 17, wherein the pressure sensor is coated with a waterproof coating.

28. The apparatus according to claim 27, wherein the waterproof coating is selected from a group consisting of Parylene, silicon, and polyurethane.

29. The apparatus according to claim 17, further comprising the irrigation-aspiration sub-system, which is configured to convey irrigation fluid along the irrigation channel at a rate of between 1 and 5 milliliters per minute.

30. The apparatus according to claim 17, further comprising:
the irrigation-aspiration sub-system configured to convey irrigation fluid along the irrigation channel intermittently providing periods of irrigation activity and intervening periods of irrigation inactivity; and
a processor configured to: sample respective values from the signal provided by the pressure sensor corresponding to time values during the periods of irrigation inactivity; and
compute pressure values responsively to the respective sampled values.

31. The apparatus according to claim 17, further comprising:
the irrigation-aspiration sub-system configured to convey irrigation fluid along the irrigation channel responsively to a modulated irrigation rate providing periods of irrigation activity above a given irrigation rate and intervening periods of irrigation activity below the given irrigation rate; and
a processor configured to: sample respective values from the signal provided by the pressure sensor corresponding to time values during the periods of irrigation activity below the given irrigation rate; and compute pressure values responsively to the respective sampled values.

32. The apparatus according to claim 17, further comprising a processor configured to:
compute a pressure value responsively to the signal provided by the pressure sensor; and
control the irrigation-aspiration sub-system responsively to the computed pressure value.

33. An ophthalmic method, comprising:
providing an ophthalmic curette apparatus, including:
a handle having a distal end;
a tube having a proximal end connected with the distal end of the handle, and having a distal tip configured to be inserted into an eye;
an irrigation channel extending from the proximal end to the distal tip of the tube; and
a pressure sensor comprising a sensing surface and disposed at the distal tip of the tube, wherein the pressure sensor is configured to be inserted into the eye and provide a signal responsively to intraocular pressure inside the eye, wherein the irrigation channel is configured to be coupled with an irrigation-aspiration sub-system to convey irrigation fluid along the irrigation channel, and wherein the irrigation channel is shaped, and positioned with respect to the pressure sensor, to direct a flow of the irrigation fluid over the sensing surface of the pressure sensor;
sampling values from the signal provided by the pressure sensor;
computing a pressure value responsively to the signal provided by the pressure sensor; and
adjusting an aspiration rate or irrigation rate responsively to the computed pressure value.

34. The method according to claim 33, wherein the adjusting includes adjusting an aspiration rate of eye fluid and waste matter from the eye via an aspiration line of a phacoemulsification probe.

35. The method according to claim 33, wherein the adjusting includes adjusting an irrigation rate of the irrigation fluid to the eye via an irrigation line of a phacoemulsification probe.

36. The method according to claim 33, wherein the adjusting includes adjusting an irrigation rate of the irrigation fluid to the eye via the irrigation channel of the ophthalmic curette apparatus.

37. The method according to claim 33, further comprising conveying irrigation fluid along the irrigation channel intermittently providing periods of irrigation activity and intervening periods of irrigation inactivity, wherein:
the sampling includes sampling respective values from the signal provided by the pressure sensor corresponding to time values during the periods of irrigation inactivity; and
the computing includes computing pressure values responsively to the respective sampled values.

38. The method according to claim 33, further comprising conveying irrigation fluid along the irrigation channel responsively to a modulated irrigation rate providing periods of irrigation activity above a given irrigation rate and intervening periods of irrigation activity below the given irrigation rate, wherein:
the sampling includes sampling respective values from the signal provided by the pressure sensor corresponding to time values during the periods of irrigation activity below the given irrigation rate; and
the computing includes computing pressure values responsively to the respective sampled values.

39. A method to manufacture an ophthalmic curette, the method comprising:
connecting a proximal end of a tube to a distal end of a handle;
providing an irrigation channel extending from the proximal end to a distal tip of the tube;
disposing a pressure sensor at the distal tip of the tube so that the pressure sensor provides a signal responsively to intraocular pressure inside the eye, and wherein the providing and the disposing are performed so that the irrigation channel is shaped, and positioned with respect to the pressure sensor, to direct a flow of irrigation fluid from the irrigation channel over a sensing surface of the pressure sensor.

40. The method according to claim 39, wherein the disposing includes disposing the pressure sensor at least partially in the irrigation channel.

41. The method according to claim 39, further comprising disposing the irrigation channel in the tube and extending from the proximal end to a distal tip of the tube, the disposing the pressure sensor includes disposing the pressure sensor at least partially in the irrigation channel at the distal tip.

42. The method according to claim 41, wherein the tube has a beveled opening, which extends longitudinally along all of the distal tip of the tube.

43. The method according to claim 42, wherein the beveled opening defines a plane which has an angle in the range of 30 to 70 degrees with a plane perpendicular to a direction of elongation of the tube.

44. The method according to claim 39, wherein the tube has a minimum length of 2 cm.

45. The method according to claim 39, wherein the tube has an outer diameter between 0.1 mm and 0.8 mm.

46. The method according to claim 39, wherein the tube has a wall thickness between 0.03 mm and 0.2 mm.

47. The method according to claim 39, further comprising coating the pressure sensor with a waterproof coating.

48. The method according to claim 47, wherein the waterproof coating is selected from a group consisting of: Parylene; silicon; and polyurethane.

49. The method according to claim 47, further comprising calibrating the pressure sensor after the coating.
